(19) United States
(12) Patent Application Publication
(10) Pub. No.: US 2011/0270409 A1
(43) Pub. Date: Nov. 3, 2011

(54) ORTHOTOPIC ARTIFICIAL BLADDER PROSTHESIS

(76) Inventor: Antonio Sambusseti, Cremona (IT)

(21) Appl. No.: 13/143,121

(22) PCT Filed: Dec. 28, 2009
(86) PCT No.: PCT/EP09/09280

§ 371 Date: Jul. 1, 2011

(30) Foreign Application Priority Data
Jan. 7, 2009 (IT) MI2009A 000004

(51) Int. Cl. A61F 2/04

(52) U.S. Cl. 623/23.65

(57) ABSTRACT

An orthotopic artificial bladder prosthesis includes an enclosure or bag or balloon and hollow elements (200; 300) for the forced connection of ureters and urethra to the bladder in the absence of sutures.

The present invention refers to an orthotopic artificial bladder prosthesis.

As is known, when a patient's bladder is affected by severe incurable disease which compromises its proper functioning, replacement of the bladder with an artificial bladder prosthesis is desirable.

Among the various solutions developed to solve the problem is replacement of the natural bladder with a compressible bladder made of soft multi-layer silicone emptying of which takes place by simply compressing the lower abdomen, as described in patent WO 2007/039159. This type of prosthesis provides a connection between the ureters and urethra and said artificial bladder through the use of sutures. However, securing with sutures is not always possible, as in the case, for example, of the ureters and/or urethra being weakened and/or thinned for congenital or pathological reasons. Furthermore, the use of sutures requires high manual skill as well as further use of time during the replacement procedure.

Object of the present invention is to overcome the drawbacks of the prior art, by providing an orthotopic artificial bladder prosthesis that is able to replace the natural bladder and which allows a considerable improvement in the patient's quality of life.

Another object of the present invention is to provide such an orthotopic artificial bladder prosthesis that does not require the use of sutures to secure the ureters and/or the urethra to said artificial bladder thus avoiding the risk of tissue necrosis.

Yet another object of the present invention is to provide such an orthotopic artificial bladder prosthesis that is easy to produce.

These objects are achieved in accordance with the invention with the characteristics listed in appended independent claim 1.

Advantageous embodiments of the invention will be apparent from the dependent claims.

The orthotopic artificial bladder prosthesis according to the invention comprises an enclosure or bag or balloon made from a multi-layer membrane of soft and elastic synthetic material, the inner layer of which is able not to deteriorate with urine whereas the outer layer is suitable to prevent fusing with the surrounding tissues. Said orthotopic prosthesis further comprises at least two holes for connecting the ureters to said prosthesis and the same number of hollow elements having a widened circular flat base and a tubular portion passing through said holes which has substantially the same inner diameter as the outer diameter of ureters such as to accomplish an interference fit without the necessity to use glues for fixing the ureters inside said tubular portion. The ureters are then inserted forcibly inside said tubular portion of the hollow element without substantially narrowing the ureters lumen.

Further characteristics of the invention will be made clearer by the detailed description that follows, referring to a purely exemplifying and therefore non limiting embodiment thereof, illustrated in the appended drawings, in which:

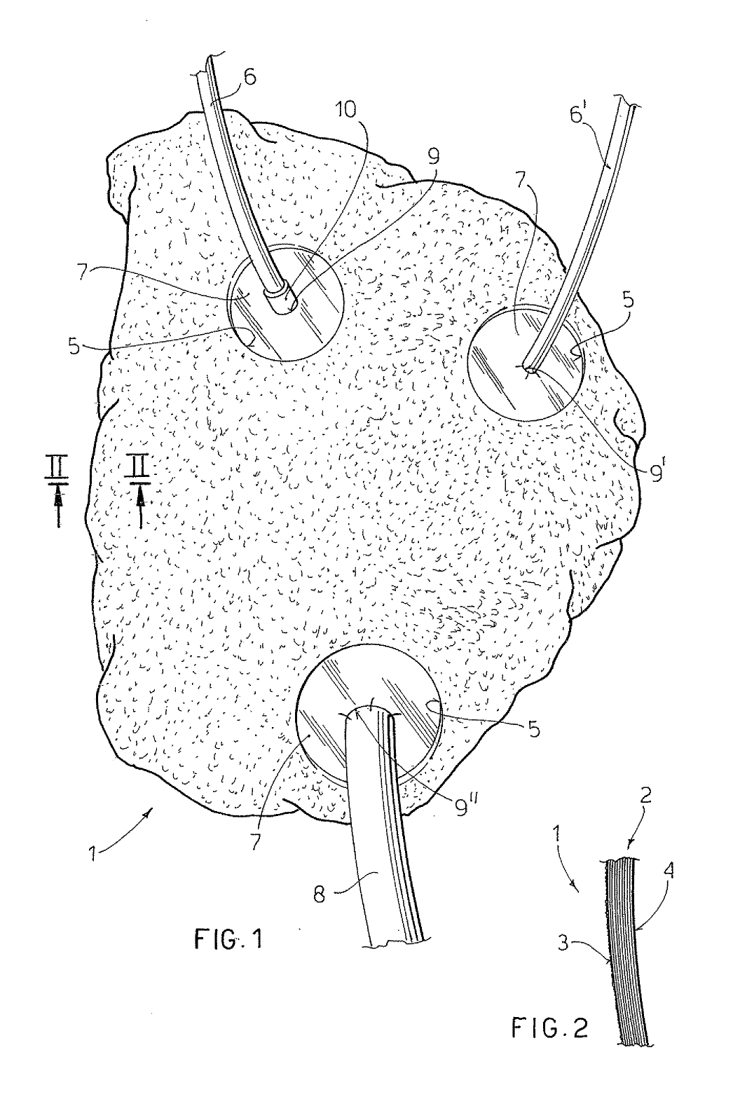

FIG. 1 is a perspective view of an orthotopic artificial bladder prosthesis in which the ureter is shown connected to said prosthesis by means of a hollow element according to the present invention whereas the other ureter and urethra are connected to the prosthesis by means of sutures according to the prior art;

FIG. 2 is an enlarged cross sectional view of a portion of the artificial bladder taken along the section II-II of FIG. 1;

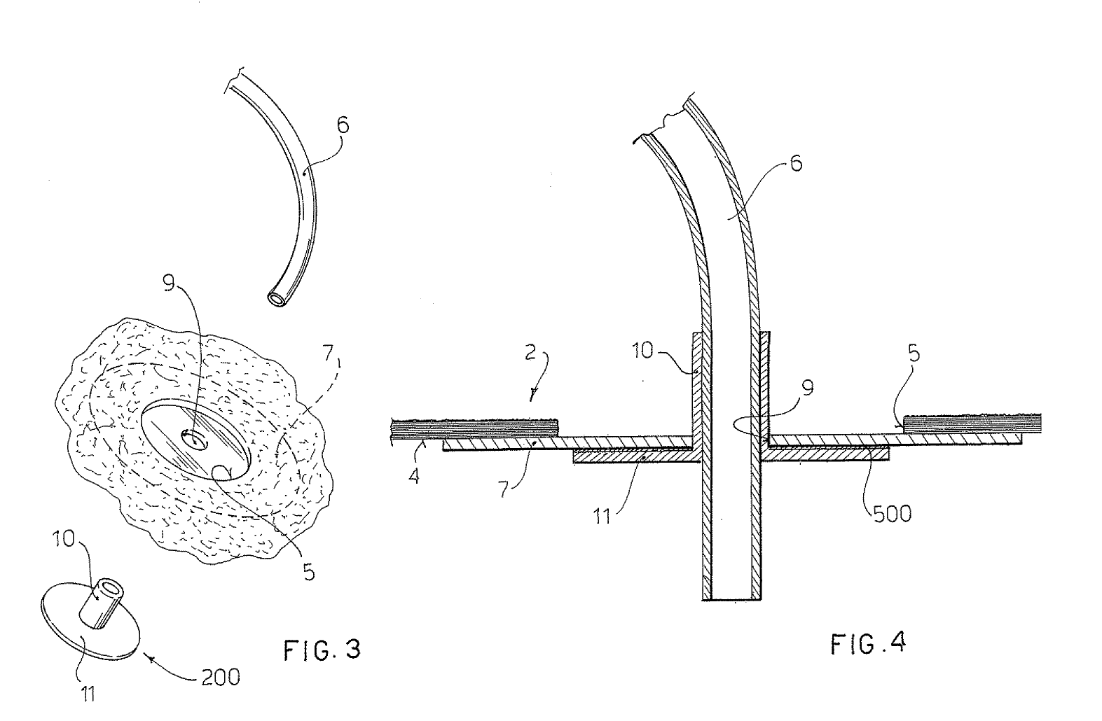

FIG. 3 is an exploded, cut-away perspective view showing the assembly between the hollow element and a ureter;

FIG. 4 is an enlarged sectional view at mid-length showing a ureter assembled in the hollow element mounted on the artificial prosthesis.

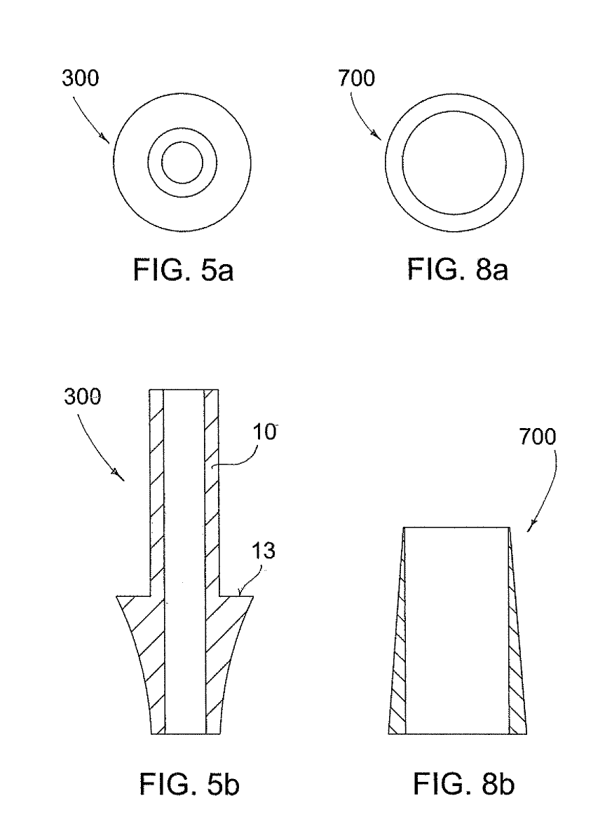

FIGS. 5a and 5b are, respectively, a top view and a mid-length sectional view of a second embodiment of the hollow element;

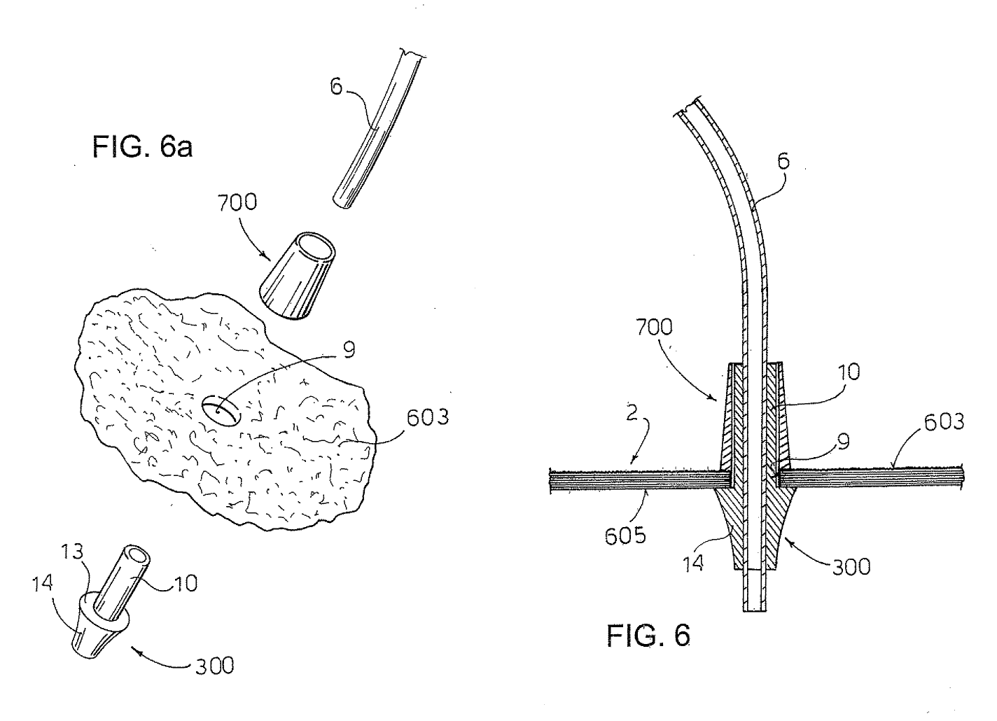

FIG. 6 is an enlarged sectional view at mid-length showing the hollow element of FIG. 5a mounted on the artificial prosthesis in which a ureter is inserted;

FIG. 6a is a perspective exploded, cut-away view showing the assembly between the hollow element of FIG. 5a and a ureter;

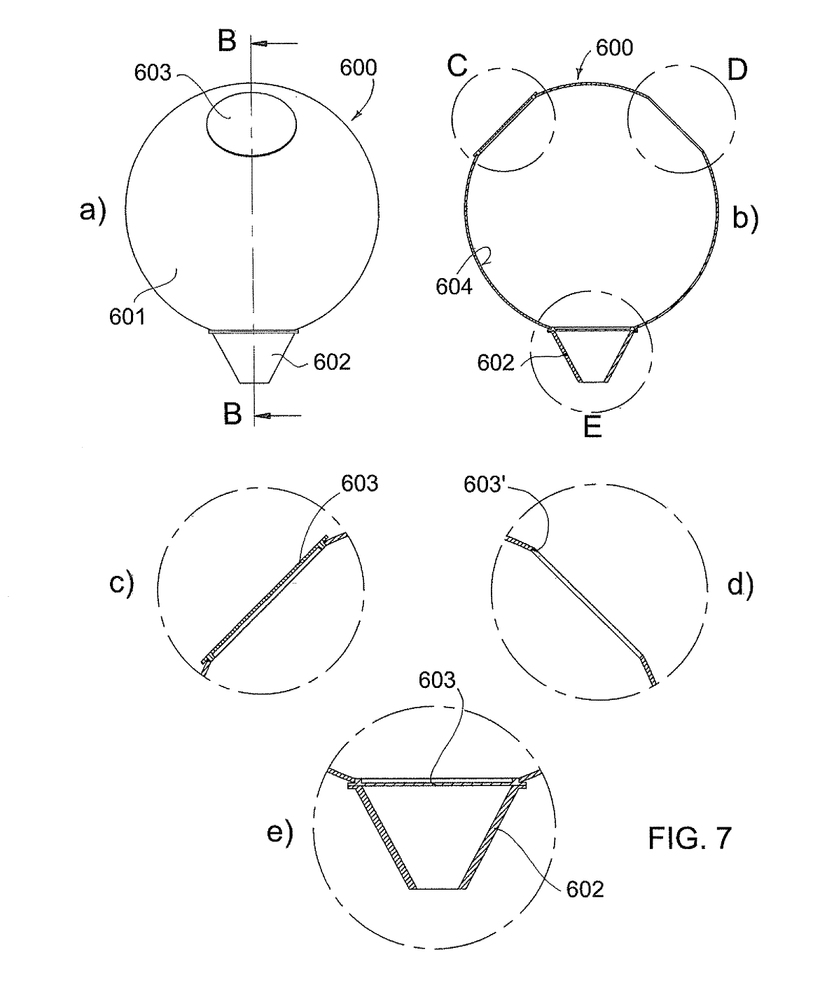

FIG. 7a is a front view of a precursor of an orthotopic artificial bladder prosthesis of a second embodiment to be coupled to the hollow element illustrated in FIGS. 5a-5b;

FIG. 7b is a sectional view of FIG. 7a taken along the line B-B;

FIGS. 7c, 7d, 7e are sectional views of the details indicated respectively with C, D and E in FIG. 7b;

FIGS. 8a e 8b are, respectively, a top view and a mid-length sectional view of a sleeve to be coupled to the hollow element of FIGS. 5a-5b.

In FIG. 1 a bladder prosthesis, designated as a whole with reference numeral 1, in the form of an enclosure or bag or collapsible balloon is illustrated. That is to say, the mechanism for filling and emptying of the prosthesis 1 works through the effect of the differences in pressure between the air inside the prosthesis and the inner liquid (urine), both inside the prosthesis 1. The capacity of said prosthesis 1 is between 100 and 900 cm³.

The prosthesis 1 of FIG. 1 consists of a multilayer membrane 2 (FIG. 2) made of soft silicone, with a thickness so as to be compressible, deflatable or collapsible. The membrane of the prosthesis preferably has a thickness of about 600 microns and consists of 20 layers of silicone, each having a thickness of about 30 microns. The silicone used can consist, for example, of copolymers of dimethyl and metavinyl siloxane, reinforced with silicon. A medical silicone is preferably used, such as, for example, that known by the code number MED 4735™ and marketed by Nusil Technology.

The outermost layer 3 of the silicone membrane 2 can be texturized so as to obtain a rough surface or it can be coated with turbostratic pyrolytic carbon, in order to reduce the risk of adhesion of the fibrous capsule to the prosthesis 1. Preferably the outermost layer 3 is coated with turbostratic pyrolytic carbon. The inner surface of the prosthesis 1, on the other hand, is coated with a microfilm 4 of highly biocompatible material, such as pyrolytic turbostratic carbon, for example, having a thickness of about 0.2-0.3 microns.

The process for obtaining said membrane is described in patent application WO 2007/039159 fully incorporated herein by reference.

The prosthesis 1 shown in FIG. 1 is also provided with three portions of membrane 7 which have a hole 9, 9′, 9″ centrally. Said portions of membrane 7 are applied to the inner surface 4 (FIG. 4) of the membrane 2 of the prosthesis 1 to cover the holes 5 previously formed on said prosthesis 1 according to what is described in WO 2007/039159.

Said portions 7 of membrane, which are of a larger size than the hole 5 (FIG. 4), are generally used in artificial bladders in order to facilitate suturing of the ureters and the urethra to the prosthesis 1. Said portions 7 of membrane can be similar to the multi-layer membrane 2 which forms the bag of the prosthesis 1 or without the texturized layer 3.

As shown in FIG. 1, according to the invention, the ureter 6 is connected to the membrane 7 by means of a tubular portion 10 inside which said ureter 6 is inserted. The tubular portion 10 forms part of a hollow element shown in FIG. 3 and designated with reference numeral 200. Said hollow element 200 also has a widened circular flat base 11 from which said tubular portion 10 begins. Said tubular portion 10 has the longitudinal axis perpendicular to said circular base 11 and has an inner diameter equal along its length as it has no inner recesses and/or projections. On the circular base 11 there is placed a silicone glue 500 (FIG. 4) which allows the hollow element 200 to be made integral with the membrane 7, in turn integral with the membrane 2 of the prosthesis 1, after the tubular portion 10 of said hollow element 200 has been inserted in the hole 9 present on the membrane 7 as illustrated in FIG. 4.

The tubular portion 10 has an inner diameter between 5 and 30 charrier (Ch) or between 5 and 15 Ch, a thickness between 1.0 and 10 mm and a length not exceeding about 5 cm. The circular base 11 has a diameter not exceeding that of the hole 5 on which the membrane 7 is applied.

The hole 9 on the membrane 7 generally has a size between 5 and 30 Ch unit and is such as to allow forcible passage of the tubular part 10 of the hollow element 200. It should be noted that 1 Ch corresponds to ⅓ mm.

The hollow element 200 inclusive of the tubular portion 10 is preferably coated, both on the inside and on the outside, with highly biocompatible biomaterial, such as for example pyrolytic turbostratic carbon.

During the surgical stage the ureter 6 is inserted forcibly inside the tubular portion 10 of the hollow element 200 which grips lightly around the tube of the ureter 6 thanks to its elasticity. The ureter is inserted inside the tubular portion 10 so as to protrude with respect to the circular base 11 of the hollow element 200 as illustrated in FIG. 4.

In this manner it is no longer necessary to fix the ureter 6 to the portions of membrane 7 by means of sutures as happens, on the other hand, in the prior art illustrated in FIG. 1 for the ureter 6′. That allows to avoid necrosis phenomena which can occur in case of sutures. In addition the interference fit between ureter and the hollow element 200 prevents to use and apply glues on ureter surface.

In another embodiment (not illustrated in the figure) of the invention the membrane 7 is absent and the hole 9 which allows the passage of the hollow element 200 is formed directly on the membrane 2 of the prosthesis 1 without having to make the hole 5 illustrated in FIG. 1, and cover it by means of the membrane 7.

Naturally, the hollow element 200, suitably sized, can also be used to connect the ureter 8 to the prosthesis 1.

Furthermore, the artificial prosthesis 1 on which the hollow elements 200 are used to secure the ureters 6, 6′ according to the present invention can be an artificial prosthesis which has a catheter or stent in place of the urethra 8.

As an alternative to the embodiment illustrated in FIG. 4, the system represented by the tubular body 200 comprising the elements 10, 11 can be replaced by an element 300 illustrated in FIGS. 5 and 6.

Said element 300 is a single piece and has a tubular portion 10 in which to insert the ureter similarly to what was described previously in relation to element 200, and a truncated conical portion 14 connected to said tubular portion 10 by means of a circular surface 13 (FIG. 5b) which has the function of allowing gluing of said element 300 to the inner surface of an artificial bladder 1. Said element 300 is made of silicone with a hardness of about 50 Shore.

In a preferred embodiment of said element 300, the tubular portion 10 has an inner diameter of about 6 mm and an outer diameter of about 10 mm, a total length of about 50 mm, of which 30 mm represent the length of the tubular part 10, and a diameter of the circular surface 13 of about 20 mm.

Said one-piece element 300 is preferably coated with pyrolitic turbostratic carbon both internally and externally, and is preferably used in combination with an artificial bladder also coated with said turbostratic carbon both internally and externally but not texturized.

Coupling of said single piece 300 as defined above to an artificial bladder or prosthesis 1, also coated with turbostratic carbon both internally and externally but not texturized, is achieved through a process having a sequence of stages which will be described hereunder.

To start with, a prosthesis in the form of a balloon 600 (FIG. 7a) consisting of a membrane 2 made of soft silicone is obtained.

Said membrane 2 can be multi-layered and made with a thickness so as to be compressible, deflatable and collapsible, for example of about 600 microns consisting for example of 20 layers of silicone, each having a thickness of about 30 microns, obtained in accordance with what is described above. Alternatively, said membrane 2 can be a single layer made by means of silicone moulding.

In a preferred embodiment said balloon 600 has a thickness of 0.6 mm and a diameter between about 72 and 74 mm.

On said balloon 600 an aperture (not illustrated) and three circular holes 603′ (FIGS. 7b and 7d) are formed, identifying three substantially flat areas, each area provided with a stopper type cover 603 (FIGS. 7a and 7b). In a preferred embodiment said holes 603′ have a diameter of about 22 mm and said stoppers 603 have an outside diameter of about 26 mm.

After said balloon 600 has been obtained, a circular conical frustum 602 (FIG. 7a), hollow on the inside and made of silicone, which faces towards the outer surface 601 of said balloon 600, is glued to coincide with one of the three stoppers 603. In a preferred embodiment the conical frustum 602 has a height of about 15 mm, a base with a diameter of about 24 mm, an inner diameter of the hole of about 6 mm and a thickness of about 1 mm.

Said conical frustum 602 serves as a guide for insertion of the urethra during the surgical stage, as will be described hereunder. The other two flattened circular areas coinciding with the two stoppers 603, without the conical frustum 602, on the other hand, will serve for coupling with above mentioned respective elements in a single piece 300 as will be described hereunder.

After the silicone balloon 600 has been obtained, a first deposition of turbostratic carbon will be made on the outer surface 601, according to the prior art, after having shielded the edges of the aperture mentioned previously, then proceeding with a first vulcanization of said balloon 600.

Once the balloon 600 provided with the cone 602 with the outer surface 601 coated with turbostratic carbon like the lateral surface of the conical frustum 602 has been obtained, said balloon is turned inside out a first time through the aperture (not shown in the figure) so as to bring the surface 601 and the cone 602 from the outside to the inside of the balloon 600.

At this point the inner surface 604 opposite the surface 601 is turned to the outside: coating of said surface 604 is then performed by means of application of turbostratic carbon after suitable shielding of the surfaces 605 of the stoppers 603 now facing outwards, and of the edges of the opening for turning inside out.

A second vulcanization is subsequently carried out, after which the shielding of the surfaces 605 (FIG. 7c) of the stoppers 603 is removed.

At this point it is possible to make a through hole 9 (or 9′) (FIGS. 6 and 6a) on one or both surfaces 605 of the stoppers 603 free of the cone 602, to allow insertion and passage in said hole 9 of the tubular portion 10 of each element 300, also previously coated with turbostratic carbon, then proceeding with gluing of the surface 13 (FIG. 5) on the surface 605 not coated with turbostratic carbon.

At the end of gluing each element 300 has its own conical portion 14 (FIG. 5b) facing towards the outside of the balloon 600 and the tubular portion 10 facing towards the inside.

Said one-piece element 300 has been coated with turbostratic carbon both internally and externally, similarly to what was done on the balloon 600 with shielding of the surface 13, making a longitudinal cut, shielding the edges of said cut, applying the turbostratic carbon on both surfaces of the opened longitudinally cut piece 300, vulcanizing, and subsequently removing the shielding of the edges of the cut to be able to carry out gluing of said two shielded edges.

After coupling of one or two elements 300 with the balloon artificial bladder 600, the balloon 600 is turned inside out again returning thus to the situation illustrated in FIGS. 6 and 7a where the surface 601, the cone 602 and the tubular portion 10 of each of said elements 300 face outwards whereas the conical portion 14 of the element 300, the surface 604 of the balloon and the surface 605 of the stoppers 603 are turned towards the inside of the balloon 600.

At this point the edges of the aperture for turning the balloon inside out can be glued together in order to obtain a closed balloon 600 and thus a closed bladder.

The above mentioned gluing operations are preferably carried out with the glue 500 previously described.

The through hole 9 (or 9′) is preferably made using a stainless steel neurosurgery stylet or a punch.

With the procedure thus far described an orthotopic prosthesis 1 of artificial bladder 600 coated both internally and externally with turbostratic carbon is obtained, with the conical frustum 602 and the tubular portion 10 of the one-piece element 300 faced outwards which is ready to be implanted in the patient.

During the operation the conical frustum 602 will be passed through by a stainless steel punch or neurosurgery stylet in order to pierce the stopper 603 beneath the conical frustum 602 and allow the urethra 8 to enter the artificial bladder whilst each ureter 6, 6′ will be forced inside said tubular portion 10 similarly to what was described previously for the hollow element 200.

In order to give greater protection to the ureter 6, 6′ inserted in said tubular portion 10 of said one-piece element 300, and a greater rigidity to said tubular portion, it is preferable also to use a sleeve 700 (FIG. 8) to be applied externally to the tubular portion 10 of the element 300 as illustrated in FIG. 6, once the ureter 6, 6′ has been inserted.

With the process thus far described it is therefore possible to obtain another embodiment of the prosthesis 1 according to the invention where the artificial bladder 1 is a non texturized balloon 600 but is coated internally and externally with pyrolytic turbostratic carbon, which further comprises a conical frustum 602 able to connect said prosthesis 1 to said urethra 8. Furthermore, said balloon provides for the hollow element for fixing of the ureters 6. 6′ to be a single piece 300 coated internally and externally with pyrolytic turbostratic carbon which is formed by a frustoconical portion 14 in addition to the widened base 13 which is connected to the tubular portion 10 which will be inserted in the through holes 9. 9′, similarly to what is reported for the element 200, whereas the widened base 13 of the element 300 will come into contact with the inside surface 605 of said membrane 2, similarly to the circular base 11 of the element 200 previously described.

Numerous changes and modifications of detail within the reach of a person skilled in the art can be made to the present embodiment of the invention without thereby departing from the scope of the invention as set forth in the appended claims.

1-13. (canceled)

14. An orthotopic artificial bladder prosthesis (1) comprising an enclosure or bag or balloon made from a multi-layer membrane (2) of soft and elastic synthetic material and at least two holes (9, 9′) for connecting the ureters (6, 6′) to said prosthesis (1) and a further hole for passage of the urethra (8) or of a stent, characterized in that fixing of said ureters (6, 6′) is provided by means of a respective hollow element (200) comprising a widened flat base (11) applied inside said membrane (2) and a tubular portion (10) passing through said hole (9, 9′) and inside which said ureter (6, 6′) is inserted in a forced manner such as to obtain an interference fit, said tubular portion (10) being substantially perpendicular to said flat base (11) and having an inner diameter equal along its length, and further characterized in that the outermost layer (3) of the silicone membrane (2) and the inner surface (4) are coated with pyrolytic turbostratic carbon and the hollow element (200) inclusive of the tubular portion (10) is coated, both internally and externally with turbostratic pyrolytic carbon.

15. The prosthesis according to claim 14, wherein said holes (9, 9′) are made in respective portions of membrane (7), said portions of membrane (7) being applied to the inner surface (4) of the membrane (2) of the prosthesis (1) to cover holes (5) previously made in said prosthesis (1).

16. The prosthesis according to claim 14, wherein a silicone glue (500) is placed on said widened base (11), said glue solidly connecting the hollow element (200) to the multi-layer membrane (2) or to the membrane (7).

17. The prosthesis according to claim 14, wherein said tubular portion (10) has an inner diameter between 5 and 30 charrier (Ch), a thickness between 1.0 and 10 mm and a length not exceeding 5 cm.

18. The prosthesis according to claim 14, wherein said hole (9) has a size generally between 5 and 30 Ch and is such as to permit forced passage of the tubular portion (10) of the hollow element (200).

19. The prosthesis according to claim 14, wherein the ureter (6, 6′) inserted in the tubular portion (10) of the hollow element (200) projects with respect to said widened base (11).

20. The prosthesis according to claim 14, wherein said multi-layer membrane (2) is made of soft silicone, with a thickness of approximately 600 microns.

21. The prosthesis according to claim 14, wherein said urethra (8) is also fixed by means of said hollow element (200).

22. The prosthesis according to claim 14 wherein said artificial bladder (1) is a non texturized balloon (600) coated internally and externally with pyrolytic turbostratic carbon, further comprising a conical frustum (602) suitable for connection of said prosthesis to said ureter (8), characterized in that the hollow element for fixing of said ureters (6, 6′) is a single piece (300) coated internally and externally with pyrolytic turbostratic carbon and comprises a frustoconical portion (14) in addition to the widened base (13) which is connected to the tubular portion (10) passing in said hole (9, 9′), said widened base (13) being in contact with a surface (605) facing towards the inside of said membrane (2).

23. The prosthesis according to claim 22 in which said surface (605) is the bottom surface of a cover (603) placed on the outer surface (601) of said balloon (600).

24. The process for preparing the prosthesis as defined in claim 22 comprising
obtaining a prosthesis in the form of a balloon (600) having an outer surface (601) and an inner surface (604), said balloon (600) consisting of a membrane (2) of soft silicone;
obtaining on said balloon an aperture and three circular holes (603′), and closing each hole (603′) with a stopper type cover (603);
gluing onto one of the covers (603) of a circular conical frustum (602) of silicone, hollow on the inside and facing towards the outer surface (601) of said balloon (600);
deposition of turbostratic carbon on the outer surface (601) of the balloon (600) and on the surface of said conical frustum (602), after shielding of the edges of said aperture;
first vulcanization;
turning inside out of said vulcanized balloon (600) through said aperture and of said surface (601) from the outside to the inside of said balloon (600) and of said surface (604) from the inside to the outside;
coating of said surface (604) by application of turbostratic carbon after suitable shielding of the surfaces (605) of the covers (603) and of the edges of the aperture;
second vulcanization and removal of the shielding;
formation of a through hole (9, 9′) on the surface (605) of said stopper (603) without the cone (602), insertion in said hole (9, 9′) of the tubular portion (10) of the element (300), previously coated with turbostratic carbon, gluing of the widened surface (13) to the uncoated surface (605),
subsequent turning inside out of said balloon (600) to turn said cone (602) and said tubular portion (10) of said element (300) towards the outside, and subsequent gluing together of the edges of the aperture for turning inside out.

25. The process according to claim 24 wherein the one-piece element (300) has been coated with turbostratic carbon both internally and externally, with the exception of the surface (13) by making a longitudinal cut, shielding the edges of said cut, applying turbostratic carbon to both surfaces of the opened element (300), vulcanization, and subsequently removal of the shielding of the edges of the cut, and subsequent gluing of said two shielded edges.

26. The process for preparing the prosthesis as defined in claim 23 comprising
obtaining a prosthesis in the form of a balloon (600) having an outer surface (601) and an inner surface (604), said balloon (600) consisting of a membrane (2) of soft silicone;
obtaining on said balloon an aperture and three circular holes (603′), and closing each hole (603′) with a stopper type cover (603);
gluing onto one of the covers (603) of a circular conical frustum (602) of silicone, hollow on the inside and facing towards the outer surface (601) of said balloon (600);
deposition of turbostratic carbon on the outer surface (601) of the balloon (600) and on the surface of said conical frustum (602), after shielding of the edges of said aperture;
first vulcanization;
turning inside out of said vulcanized balloon (600) through said aperture and of said surface (601) from the outside to the inside of said balloon (600) and of said surface (604) from the inside to the outside;
coating of said surface (604) by application of turbostratic carbon after suitable shielding of the surfaces (605) of the covers (603) and of the edges of the aperture;
second vulcanization and removal of the shielding;
formation of a through hole (9, 9′) on the surface (605) of said stopper (603) without the cone (602), insertion in said hole (9, 9′) of the tubular portion (10) of the element (300), previously coated with turbostratic carbon, gluing of the widened surface (13) to the uncoated surface (605),
subsequent turning inside out of said balloon (600) to turn said cone (602) and said tubular portion (10) of said element (300) towards the outside, and subsequent gluing together of the edges of the aperture for turning inside out.
